(19) United States
(12) Patent Application Publication
(10) Pub. No.: US 2025/0250524 A1
(43) Pub. Date: Aug. 7, 2025

(54) SYSTEMS AND METHODS FOR RECYCLING GROWTH FACTORS AND OTHER COMPONENTS

(71) Applicant: Terasaki Institute for Biomedical Innovation, Los Angeles, CA (US)

(72) Inventors: Alireza Khademhosseini, Los Angeles, CA (US); Seyedvahid Hosseini, Newbury Park, CA (US); Sayed Alireza Hassani Najafabadi, Encino, CA (US); Reza Abbasgholizadeh, Los Angeles, CA (US)

(73) Assignee: Terasaki Institute for Biomedical Innovation, Los Angeles, CA (US)

(21) Appl. No.: 18/855,701

(22) PCT Filed: Apr. 13, 2023
(86) PCT No.: PCT/US2023/018432

§ 371 Date: Oct. 10, 2024

Related U.S. Application Data
(60) Provisional application No. 63/330,824, filed on Apr. 14, 2022.

(51) Int. Cl. C12M 1/00; C12N 5/077

(52) U.S. Cl. CPC C12M 29/04; C12M 23/40; C12M 29/18; C12N 5/0658

(57) ABSTRACT

The present disclosure relates, in certain aspects, to the production of cell-based meat and/or other cellular products, for example, in bioreactors. In some cases, the production of these may be sustainable. In addition, certain embodiments are directed to reducing operational costs by reducing the demand for media. For example, some aspects are generally directed to systems and methods for retaining growth factors, proteins, or other components, for example, by passing fluid from a bioreactor through one or more filters to retain the growth factors or other components and return them to the bioreactor, while removing other components (e.g., waste products or smaller molecules) from the bioreactor. In some cases, this may include filtration using a filter with a molecular weight cut-off of between 1 kDa and 10 kDa, e.g., to retain growth factors, proteins, or other components.

### RELATED APPLICATIONS

This application claims the benefit of U.S. Provisional Patent Application Ser. No. 63/330,824, filed Apr. 14, 2022, entitled “Systems and Methods for Recycling Growth Factors and Other Components,” incorporated herein by reference in its entirety.

### FIELD

The present disclosure relates, in certain aspects, to the production of cell-based meat and/or other cellular products, for example, in bioreactors. In some cases, the production of these may be sustainable. In addition, certain embodiments are directed to reducing operational costs by reducing the demand for media.

### BACKGROUND

Global production and consumption of meat continue to surge as demand is driven upward by population growth, individual economic gain, and urbanization. This rising demand is problematic, as current methods of large-scale animal husbandry are linked to public health complications, environmental degradation, and animal welfare concerns. With regard to human health, the animal agriculture industry is interconnected with foodborne illness, diet-related disease, antibiotic resistance, and infectious disease. Notably, zoonotic diseases (e.g., Nipah virus, influenza A, etc.) have been linked to agricultural intensification. Animal agriculture also contributes to environmental issues including greenhouse gas emissions, land use, and water use. The United Nations Intergovernmental Panel on Climate Change released a 2018 report asserting that greenhouse gas emissions must be reduced 45% by 2030 to prevent global temperatures from increasing 1.5° C.; a target that could mitigate catastrophes associated with a 2.0° C. increase. Conventional mitigation techniques include improvements in reforestation, soil conservation, waste management as well as tax policy, subsidies, and zoning regulations. While these strategies remain important, the urgency of climate change may require more transformative approaches. Lastly, with regard to animal welfare concerns, each year billions of animals are killed or suffer either directly (e.g., farm animal slaughter, seafood fishing, etc.) or indirectly (e.g., fishing by-catch, wildlife declines due to habitat destruction) in relation to human food systems.

The majority of the aforementioned issues can be attributed to the fact that the raw material inputs (i.e., animals) for conventional meat production are inherently unsanitary and inefficient. Growing livestock and other animals consumed for food to maturity that they can be slaughtered to provide meat is a time consuming and expensive process.

Cell-based cultivated meat is a recent innovation in the food industry. Cell-based meat is manufactured using animal cells in vitro and in culture medium to create a meat without raising and slaughtering animals in traditional ways. Cultivated meat or cell-based meat is an alternative source of meat to replace animal-based meat. Cell-based meat is projected to be common in the global market in a few years, although one of major challenge is the high costs associated with the production of cultivated meat. For example, large scale proliferation of mammalian cells known to be expensive procedure that demand high capital expenditure at a scale that can substitute conventional meat. Accordingly, improvements are needed.

### SUMMARY

The present disclosure relates, in certain aspects, to the production of cell-based meat and/or other cellular products, for example, in bioreactors. In some cases, the production of these may be sustainable. In addition, certain embodiments are directed to reducing operational costs by reducing the demand for media. The subject matter of the present disclosure involves, in some cases, interrelated products, alternative solutions to a particular problem, and/or a plurality of different uses of one or more systems and/or articles.

For example, in one set of embodiments, techniques such as ultrafiltration may be used to capture or retain growth factors or other components, e.g., including those having significant value, while releasing other molecules, such as unused nutrients, waste products such as vitamins, minerals, lactic acid, ammonia, or the like.

In one aspect, cells may be separated from a culture media using one or more filtration devices. Certain components such as cells, proteins, growth factors, or other components may be retained, while smaller molecules, such as waste products, unused nutrients, etc. may be filtered out. In some cases, these may be sent to waste, sent to a downstream recycling system, etc. In certain embodiments, the volume lost due to the filtering out of such smaller molecules may be made up using fresh media. In addition, in some embodiments, proteins, growth factors, other components, etc. that are lost due to filtration may be determined and made up using fresh media.

In addition, certain aspects are generally directed to a device. The device, in one set of embodiments, comprises a bioreactor; a first fluidic connection connecting the bioreactor to an inlet of a first filtration device, the first filtration device further comprising a first retentate outlet and a first filtrate outlet; a second fluidic connection connecting the first filtrate outlet to the inlet of a second filtration device comprising a separator having an average molecular weight cutoff of at least 1 kDa, the second filtration device further comprising a second retentate outlet and a second filtrate outlet; a third fluidic connection connecting the second retentate outlet with a first inlet of a mixing device, the mixing device further comprising a second inlet and an outlet; and a fourth fluidic connection connecting the outlet of the mixing device with the bioreactor.

Another aspect is generally directed to a method. The method, in accordance with one set of embodiments, comprises separating a fluid from a bioreactor containing a cell-based meat culture, using a first filtration device, into a first retentate and a first filtrate, wherein the first filtrate is substantially free of cells; separating the first filtrate, using a second filtration device having an average molecular weight cut-off of at least 1 kDa, into a second retentate and a second filtrate; mixing the second retentate with fresh cell culture media to form a mixture; and returning the mixture to the bioreactor.

The method, in another set of embodiments, comprises separating a fluid from in a bioreactor, using a first filtration device, into a first retentate and a first filtrate, wherein the first filtrate is substantially free of cells; separating the first filtrate, using a second filtration device having an average molecular weight cut-off of at least 1 kDa, into a second retentate and a second filtrate; and mixing the second retentate with fresh cell culture media to form a mixture.

In still another set of embodiments, the method comprises removing fluid comprising non-human cells and growth factors from a bioreactor containing a cell-based meat culture; separating the non-human cells from the fluid; returning the non-human cells to the bioreactor; separating the growth factors from the fluid; and returning the growth factors to the bioreactor.

In another aspect, the present disclosure encompasses methods of making one or more of the embodiments described herein, for example, bioreactors for reducing the demand for media. In still another aspect, the present disclosure encompasses methods of using one or more of the embodiments described herein, for example, bioreactors for reducing the demand for media.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates a system including a bioreactor according to one embodiment;

FIG. 2 illustrates the retention of growth factors of IGF-1 and FGF, according to another embodiment;

FIG. 3 illustrates the efficacy of removal of lactic acid using filtration techniques, in accordance with yet another embodiment;

FIG. 4 illustrates cell proliferation of myoblasts in accordance with still another embodiment; and

FIG. 5 illustrates cell proliferation of myoblasts, in yet another embodiment.

### DETAILED DESCRIPTION

The present disclosure relates, in certain aspects, to the production of cell-based meat and/or other cellular products, for example, in bioreactors. In some cases, the production of these may be sustainable. In addition, certain embodiments are directed to reducing operational costs by reducing the demand for media. For example, some aspects are generally directed to systems and methods for retaining growth factors, proteins, or other components, for example, by passing fluid from a bioreactor through one or more filters to retain the growth factors or other components and return them to the bioreactor, while removing other components (e.g., waste products, unused nutrients, smaller molecules, etc.) from the bioreactor. In some cases, this may include filtration using a filter with a molecular weight cut-off of at least 1 kDa, e.g., between 1 kDa and 10 kDa, e.g., to retain growth factors, proteins, or other components.

Thus, one aspect as discussed herein is generally drawn to systems and methods for retaining growth factors, proteins, or other components, e.g., in a cell cultivation system, for example, including one or more bioreactors. Such cultivation systems may be used to cultivate meat (e.g., for food) or other products. However, as previously discussed, some components used to cultivate the cells may be relatively expensive. Non-limiting examples of such components include growth factors, proteins, etc. Examples include, but are not limited to, FGF, transferrin, albumin, signaling proteins, hormones, extracellular proteins, fibronectin, etc. As discussed herein, by reducing the loss of such components in the system, and instead retaining such components, the overall costs for cultivating cells within such a cell cultivation system may be reduced.

In one set of embodiments, components such as growth factors, proteins, etc. may be present within a bioreactor, e.g., within a fluid. Such components may be, e.g., dissolved or suspended within the fluid. However, the fluid may also contain smaller molecules, which in some cases may be undesirable to retain within the bioreactor. These include, for example, cellular waste products such as lactic acid, ammonia, dead cells, ethanol, lactose, CO₂, etc. It is often desirable to remove such waste products from the bioreactor, e.g., as such waste products can often have an adverse effect on cells contained with within the bioreactor, especially if allowed to build up towards higher concentrations. Accordingly in some embodiments, fluid within the bioreactor may be processed as discussed herein, e.g., to retain components such as growth factors, proteins, etc., while removing waste products from the cultivation system. For example, fluid may be removed from the bioreactor and passed through one or more filtration devices to retain larger components such as growth factors, proteins, etc. while removing the waste products.

While many other bioreactor applications also may require the removal of waste products from the fluid in the bioreactor, in many such applications (e.g., during the production of pharmaceuticals), the components of the fluid are not usually returned to the bioreactor, as this may substantially increase complexity and cost of the bioreactor and the cell cultivation system, as well as creating additional sources of contamination. Accordingly, in most bioreactor applications, no recycling of components such as growth factors, proteins, etc. is performed.

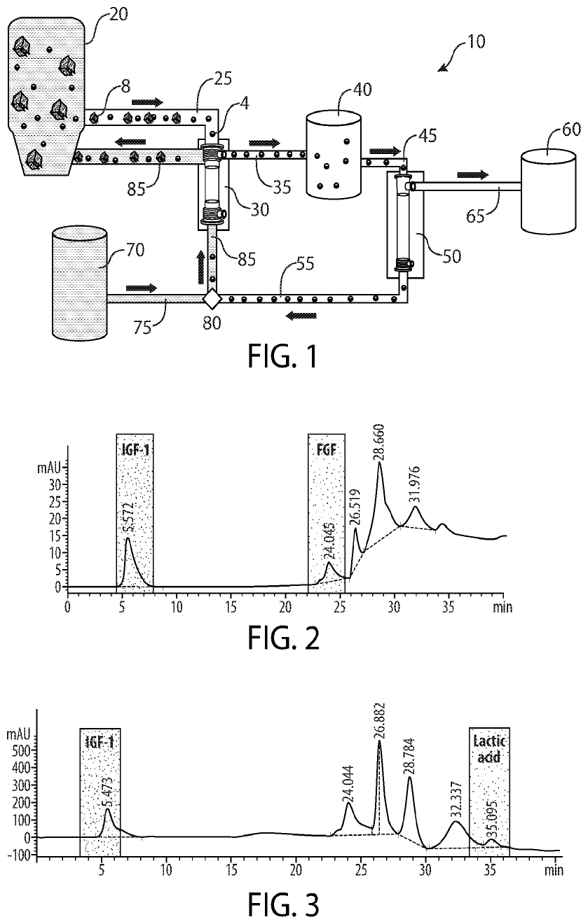

Certain embodiments are generally directed to retaining components such as proteins, growth factors, etc., e.g., in a bioreactor, for example, to aid cellular proliferation and generating cell mass. In contrast, in many industries, the generation of cell mass is considered as waste, and any cell mass is often disposed of after extraction and purification of a defined protein. One non-limiting example of a cultivation system for retaining such components is shown in FIG. 1. This figure shows system 10 with bioreactor 20, containing cells 8 and cell culture media for the cells, as well as waste products 4 produced by the cells. The cells may be cultivated within bioreactor 20 to produce cultivated meat (e.g., for food), or other products. Fluid from the bioreactor is passed through fluidic connection 25 (for example, a pipe) to filtration device 30, as indicated by the directional arrows.

In this example, filtration device 30 is used to prevent the loss of cells from the bioreactor. For instance, filtration device 30 may comprise an filtration device having a filter or separator having an average pore size of around 0.2 micrometers, or other sizes as discussed herein. Filtration device 30 may be used to separate the incoming fluid from the bioreactor into a filtrate (e.g., that is substantially free of cells from the bioreactor) and a retentate (e.g., containing cells from the bioreactor). The retentate containing cells may be returned to the bioreactor via fluidic connection 85 (or another suitable connection), while the retentate exits via fluidic connection 35. Accordingly, the cells may be retained within the bioreactor while waste products are removed.

In this example figure, the retentate than passes through fluidic connection 35 to filtration device 50 via optional reservoir 40 and fluidic connection 45. Filtration device 50 may be used to separate components such as growth factors, proteins, etc. from waste products 4 such as lactic acid, ammonia, CO₂, etc. For example, filtration device 50 may be used to separate the incoming fluid into a filtrate and a retentate. For example, filtration device 50 may be an ultrafiltration device with a separator having an average molecular weight cut-off of 5 kD, or other cut-offs such as those described herein. From filtration device 50, the filtrate (e.g., containing waste products, which are typically smaller in size) can then be passed to waste (e.g., as shown with reservoir 60) via fluidic connection 65, while the retentate (e.g., containing larger components, such as growth factors, proteins, etc.) exits filtration device 50 via fluidic connection 55.

In one set of embodiments, this retentate may optionally be combined with fresh media, e.g., as supplied from reservoir 70 via fluidic connection 75. These can be mixed together at mixer 80 and the mixture re-introduced to bioreactor 20 via fluidic connection 85. However, it should be understood that in other embodiments, no fresh media may be supplied, e.g., reservoir 70 and mixer 80 may not be present. In addition, in some instances, retentate from filtration device 30 (e.g., containing cells) may also be added to the fluid within fluidic connection 85, although in other cases, these may be kept separate. The amount of additional fresh fluid from reservoir 70 may be constant, or may be varied, for example, based on the concentration of components such as growth factors, proteins, etc. in the retentate, which can be determined using suitable sensors. In some cases, the amount of fresh media that is added may be added to produce a desired concentration or quality of media that is returned to bioreactor 20.

The above discussion is a non-limiting example of one embodiment of the present invention generally directed to cell cultivation systems for retaining growth factors, proteins, or other components. However, other embodiments are also possible. Accordingly, more generally, various aspects of the invention are directed to various systems and methods for the production of cell-based meat and/or other cellular products, for example, in cell cultivation systems, e.g., by reducing operational by reducing the demand for media.

For example, in certain aspects, a bioreactor contained within a cell cultivation system, e.g., as discussed herein, may be used to produce a cell-based meat or a cultivated meat product. Such cultivated meat products are generally intended to be consumed as food. As used herein, cell-based meat is synonymous with clean meat, cultivated meat, cultured meat, cellular meat, slaughter-free meat, and synthetic meat, among other related terms. Cultivated meat products are typically produced using in vitro cell culture or bioreactors, as opposed to “regular” meat that is grown and harvested from live animals (usually killing the animal in the process). For cultivated meat, such products are produced using cells taken from an animal, but then the cells are cultured or cultivated in vitro, e.g., in a bioreactor. This is in stark contrast to traditional techniques of sacrificing animals and harvesting meat or other organs (e.g., skin, internal organs, etc.) for food or other uses. Although the original cells seeded to form the product may have originated or otherwise have originally been derived from a living animal, the bulk of the cells forming the actual product were grown or cultured in an in vitro setting, rather than naturally as part a living animal.

A variety of products may be formed from cells cultured in vitro. For instance, certain embodiments are directed to cultivated meat products. It will be appreciated that, because it is to be eaten, such products will often be formed of edible or digestible materials, e.g., materials that can be digested, or degraded to form generally nontoxic materials within the digestive system. For instance, the clean meat may contain non-human or animal-derived cells (e.g., derived from chicken, beef, pork, mutton, goat, venison, fish, duck, turkey, shrimp, or other animals that are commonly recognized for widespread human consumption), such as muscle cells, fat cells, blood cells, or the like. In addition, in some embodiments, the clean meat product may contain other edible materials, such as plant-originated materials. Techniques involving such cultivated meat products include those described in U.S. Ser. No. 63/279,617, filed Nov. 15, 2021, entitled “Constructs Comprising Fibrin or Other Blood Products for Meat Cultivation and Other Applications,” by Hosseini, et al.; U.S. Ser. No. 63/279,631, filed Nov. 15, 2021, entitled “Methods and Systems of Preparing Cultivated Meat from Blood or Cellular Biomass,” by Hosseini, et al.; U.S. Ser. No. 63/279,642, filed Nov. 15, 2021, entitled “Systems and Methods of Producing Fat Tissue for Cell-Based Meat Products,” by Hosseini, et al.; and U.S. Ser. No. 63/279,644, filed Nov. 15, 2021, entitled “Production of Heme for Cell-Based Meat Products,” by Hosseini, et al., each of which is incorporated herein by reference in its entirety.

However, other products may also be produced in a bioreactor. For instance, the bioreactor may be used to produce a product cultivated from animal-derived cells, but the product is not necessarily one that is intended to be eaten. The cells may be human or non-human. For instance, cells from an animal may be cultured in a bioreactor to form various organs that can be harvested, such as skin, hair, fur, or the like. Thus, as a non-limiting example, leather, cultivated fur, etc. can be formed by growing cells in culture, for example as discussed herein, without the traditional method of sacrificing animals to harvest their skin or other organs.

Those of ordinary skill in the art will be familiar with techniques for culturing cells in a bioreactor. Typically, the bioreactor will contain a fluid that is used to nourish the cells and keep them alive within the bioreactor. The fluid may contain cell culture media, for example, DMEM, MEM, RPMI, or the like. A variety of cell culture media will be known to those of ordinary skill in the art. The fluid may contain components such as amino acids, vitamins, carbohydrates, inorganic salts, elements (e.g., iron, potassium, magnesium, zinc, etc.), serum, hormones, buffers (e.g., to regulate pH), antibiotics, proteins (for example, recombinant, non-recombinant, etc.), growth factors, or the like. In some cases, the fluid may contain a platelet lysate and/or a platelet rich plasma. Sec, e.g., Int. Pat. Apl. Ser. No. PCT/US22/19628, entitled “Growth Factors for Laboratory Grown Meat and Other Applications,” filed on Mar. 9, 2022, incorporated herein by reference in its entirety.

In addition, in some cases, the bioreactor may contain cell scaffolding material, e.g., as discussed in U.S. Ser. No. 63/159,403, filed Mar. 10, 2021, entitled “Constructs for Meat Cultivation and Other Applications,” by Khademhosseini, et al., incorporated herein by reference in its entirety. The cell scaffolding material may contain grooves in some embodiments. In certain cases, the cell scaffolding material may comprise a plant-originated material, such as a plant-originated protein. Such plant-originated materials may be harvested directly from a plant, be grown in vitro (e.g., in cell culture from a culture initially originating in a plant), be synthetically produced (e.g., without using a plant, e.g., chemically produced), etc. Examples of protein-originated material include, but are not limited to, cellulose or certain proteins, such as prolamin, zein, fibrin, gliadin, hordein, secalin, kafirin, avenin, gliadine, 2S albumin, globulin, glutelin, etc. The plant that material originates from may be any plant, including but not limited to food crop plants. Non-limiting examples of plants include, but are not limited to, wheat, barley, rye, corn, sorghum, oats, quinoa, hemp, potato, soy, etc.

The bioreactor may be controlled to allow cell growth and cultivation to occur, e.g., using suitable techniques known by those of ordinary skill in the art. In some cases, one or more factors may be controlled, e.g., to promote cell growth within the bioreactor. Suitable factors that may be controlled include, but are not limited to, pH, temperature, cell density, humidity, pressure, nutrients (e.g., as discussed above), agitation, gas concentrations (e.g., of O₂, CO₂, etc.), waste product concentrations, or the like.

For example, in some cases, the fluid may include one or more growth factors or other proteins, e.g., to facilitate the growth of cells within the bioreactor. These may be added artificially, and/or may be expressed by some of the cells within the bioreactor, etc. For instance, as discussed herein, it may be desirable to retain some or all of the growth factors, e.g., while cultivating cells within a cell cultivation system having the bioreactor. Examples of growth factors include, but are not limited to, epidermal growth factor, fibroblast growth factor, vascular endothelial growth factor, or the like. Other examples of growth factors and related proteins include, but are not limited to, adrenomedullin, angiopoietin, autocrine motility factor, bone morphogenetic protein, ciliary neurotrophic factor family, ciliary neurotrophic factor, leukemia inhibitory factor, macrophage colony-stimulating factor, granulocyte colony-stimulating factor, granulocyte macrophage colony-stimulating factor, epidermal growth factor, ephrins, erythropoietin, fibroblast growth factor, fetal bovine somatotrophin, GDNF ligands, neurturin, persephin, artemin, growth differentiation factor-9, hepatocyte growth factor, hepatoma-derived growth factor, insulin-like growth factor, insulin-like growth factor-1, insulin-like growth factor-2, interleukins, keratinocyte growth factor, migration-stimulating factor, macrophage-stimulating protein, myostatin, neuregulins, brain-derived neurotrophic factor, nerve growth factor, neurotrophins, placental growth factor, platelet-derived growth factor, renalase, T-cell growth factor, thrombopoietin, transforming growth factor alpha, transforming growth factor beta, tumor necrosis factor-alpha, vascular endothelial growth factor, or the like. Many such growth factors can be readily obtained commercially. In some cases, the composition of such growth factors within a bioreactor may not be known with precision; for example, expression levels of such growth factors may vary, some of the growth factors may be difficult to accurately characterize, etc.

In addition, other proteins or other components (e.g., macromolecules such as transferrin, albumin, etc., including those discussed herein) may be present as well within the fluid that can facilitate cell cultivation. For example, in some cases, one or more antibodies may be present within a fluid, e.g., to kill or inhibit growth of pathogens in the bioreactor. Other non-limiting examples of components which may be desired to be retained within a cell cultivation system, e.g., having a bioreactor, include blood platelets, blood plasma, immune cells, plasma proteins, recombinant growth factors and proteins, etc. See, for example, Int. Pat. Apl. Ser. No. PCT/US22/19618, entitled “Animal-Derived Antimicrobial Systems and Methods,” filed on Mar. 9, 2022, incorporated herein by reference in its entirety. Accordingly, it may be desirable in certain embodiments to retain some or all of these within the bioreactor, e.g., while cultivating cells within a bioreactor system.

In one aspect, for example, fluid from a bioreactor may be directed to a filtration device, for example, via a suitable fluidic connection, e.g., a pipe or other conduit. The filtration device may be used to prevent or minimize the loss of cells from the bioreactor. For example, a filtration device may be used to separate or divide the incoming fluid into a first fluid and a second fluid, e.g., a filtrate and a retentate. For instance, the filtration device may comprise a separator or other device that prevents cells from passing through (forming the retentate), while allowing other components (e.g., waste products, growth factors, proteins, other components, some fluid, etc.) to pass through (forming the filtrate). Thus, for example, at least 90%, at least 95%, at least 97%, at least 98%, at least 99%, at least 99.5%, etc. of the cells entering the filtration device may exit the filtration device via the retentate (e.g., to be returned to the bioreactor), while the filtrate may be substantially or completely free of cells from the bioreactor. It should thus be understood that no filtration device is perfect, and some minor amounts of contamination of the retentate and/or the filtrate may occur. Any suitable filtration device may be used, for example, a tangential flow filter, a crossflow filter, a membrane filter, a centrifugal filter, or the like.

In some embodiments, for example, the separator may have an average pore size of 0.2 micrometers, 0.25 micrometers, etc. In certain embodiments, the separator may have an average pore size of at least 0.05 micrometers, at least 0.1 micrometers, at least 0.15 micrometers, at least 0.2 micrometers, at least 0.25 micrometers, at least 0.3 micrometers, at least 0.35 micrometers, at least 0.4 micrometers, at least 0.45 micrometers, at least 0.5 micrometers, etc. In addition, in some embodiments, the separator may have an average pore size of no more than 0.5 micrometers, no more than 0.45 micrometers, no more than 0.4 micrometers, no more than 0.35 micrometers, no more than 0.3 micrometers, no more than 0.25 micrometers, no more than 0.2 micrometers, no more than 0.15 micrometers, no more than 0.1 micrometers, etc. In addition, combinations of any of these are also possible in various embodiments; for example, the pore size may be between 0.1 micrometers and 0.3 micrometers, between 0.2 micrometers and 0.4 micrometers, between 0.25 micrometers and 0.45 micrometers, etc.

In addition, other separation techniques besides filtration may be used in the filtration device. Non-limiting examples include centrifugation-based approaches, gravity-based approaches, density gradient separation, sedimentation, microfluidic cell sorting techniques (e.g., deterministic lateral displacement), or the like.

In some aspects, the filtrate may be directed to a second filtration device, e.g., via a suitable fluidic connection. This second filtration device may be used to separate components such as growth factors, proteins, etc. from smaller molecules such as unused nutrients, waste products such as lactic acid, ammonia, ethanol, lactose, CO₂, or the like. The filtration device may separate the incoming fluid into a first fluid enriched in components such as growth factors, proteins, etc. (e.g., as the retentate), and a second fluid depleted in such components. This separation may be performed, for example, on the basis of size, charge, or other suitable factors.

The filtrate from the second filtrate device, e.g., smaller molecules such as unused nutrients, waste products, etc., may be directed to waste, a reservoir (e.g., for later recycling, disposal, etc.), or the like, e.g., via a suitable fluidic connection. In some cases, the filtrate exits the cell cultivation system. The retentate may optionally be processed before being returned to the bioreactor, e.g., as discussed herein.

It should be understood that the filtration device need not be perfect, e.g., some desired components may still be separated out (e.g., within the filtrate) and sent to waste, etc., and/or some undesired products may be retained (e.g., within the retentate), and retained within the cell cultivation system. Thus, some minor amounts of contamination of the retentate and/or the filtrate may occur.

In certain embodiments, the filtration device may comprise a separator or other device that is able to separate components on the basis of size. For example, the filtration device may comprise an ultrafiltration separator. Such separators are typically determined using a molecular weight cut-off, and are readily available commercially. For example, the filtration device may have an average molecular weight cut-off of 1 kDa, 5 kDa, or 10 kD. In addition, in certain embodiments, the separator may have an average molecular weight cut-off of at least 0.5 kDa, at least 1 kDa, at least 2 kDa, at least 3 kDa, at least 5 kDa, at least 7 kDa, at least 10 kDa, at least 15 kDa, at least 20 kDa, at least 30 kDa, at least 40 kDa, at least 50 kDa, at least 75 kDa, at least 100 kDa, etc. In some case, the separator may have an average molecular weight cut-off of no more than 100 kDa, no more than 75 kDa, no more than 50 kDa, no more than 40 kDa, no more than 30 kDa, no more than 20 kDa, no more than 15 kDa, no more than 10 kDa, no more than 7 kDa, no more than 5 kDa, no more than 4 kDa, no more than 3 kDa, no more than 2 kDa, no more than 1 kDa, etc. In addition, combinations of any of these are also possible in various embodiments. For example, the separator may have an average molecular weight cut-off of between 1 kDa and 10 kDa, between 7 kDa and 15 kDa, between 10 kDa and 20 kDa, etc.

In addition, other techniques may also be used in other embodiments, e.g., to separate components such as growth factors, proteins, etc. from smaller molecules such as unused nutrients, waste products, or the like Non-limiting examples of such techniques include cellulose membrane concentrators, dialysis, preexcitation, chromatography, charge-separation techniques, isoelectric focusing, or the like.

In some cases, at least 50 mol % of components such as growth factors, proteins, etc. may be retained by the filtration device, e.g., within the retentate. In addition, in some cases, at least 55 mol %, at least 60 mol %, at least 65 mol %, at least 70 mol %, at least 75 mol %, at least 80 mol %, at least 85 mol %, at least 90 mol %, at least 95 mol %, etc. of such components may be retained by the filtration device, e.g., within the retentate.

The retentate may, in some aspects, be returned to the bioreactor, e.g., using a suitable fluidic connection, e.g., a pipe or other conduit. The retentate may be returned directly in some cases, although in certain embodiments, fresh media may be added to the retentate prior to being returned to the bioreactor, e.g., using a mixer or other suitable device. The mixer may include, for example, a surge tank, a reservoir, a connection of two or more fluidic connections, or the like. In some cases, the media may be actively mixed, e.g., using blades, baffles, or the like. However, in some cases, the media may be passively mixed, or not mixed. After mixing, the mixture may be returned to the bioreactor, e.g., using a suitable fluidic connection.

The fresh culture media that is added may be the same or different than the fluid within the bioreactor. The fresh media may include media such as saline, DMEM, MEM, RPMI, or the like. In some embodiments, the fresh media may be provided from a suitable source of media, for example, a reservoir. In some cases, a fixed amount or flow rate of media may be used, although in certain embodiments, the amount of fresh media may be controlled in some fashion. For example, one or more sensors may be used to determine the concentration of growth factors, proteins, or other components returning to the bioreactor, which may be used to determine a make-up amount or volume of fresh media to be added (for example, by the use of fresh media containing additional growth factors, proteins, or other components).

It should be understood that the present invention is not limited to only the precise arrangement of filtration devices, fluidic connections, etc., as is described above or shown in FIG. 1. Those of ordinary skill in the art may, for example, incorporate other devices, such as additional sensors, surge tanks, sources of fluids or reagents, other fluidic network connections (e.g., valves), etc., in other embodiments of the invention. As a specific example, reservoir 40 in FIG. 1 may not necessarily be present in certain embodiments, and/or other reservoirs may be added, etc.

In some aspects, the bioreactor may optionally contain one or more microcarriers or other types of scaffolds, e.g., for cultivated cells. In some cases, the microcarriers may have one or more grooves defined therein. These may be useful, for example, to allow cells such as myoblasts to orient in and thereby become aligned, e.g., when cultured. In this way, myotubes generally aligned with the grooves may be formed as the myoblasts fuse together to form the myotubes. In some cases, the microcarriers or other scaffolds may be formed from materials that are edible, for example, plant-originated materials such as plant-originated proteins. Non-limiting examples of plant-originated proteins that may be used as microcarriers or scaffolds include gliadin, secalin, zein, kafirin, avenin, or the like. In addition, in some cases, other materials can be used, e.g., instead of and/or in addition to plant-originated materials, for example, polymers, carbohydrates, sugars, saccharides, etc. However, it should be understood that in other embodiments, other applications for cultivated animal-derived products are possible, including applications that are not intended for food consumption. Thus, in certain embodiments, the scaffolds or microcarriers need not be formed from materials that are edible and/or degradable. See, for example, U.S. Ser. No. 63/159,403, filed Mar. 10, 2021, entitled “Constructs for Meat Cultivation and Other Applications,” by Khademhosseini, et al, incorporated herein by reference in its entirety.

In addition, in some cases, the bioreactor may contain microcarriers or other scaffolds comprising fibrin. Non-limiting examples of these may be found in U.S. Ser. No. 63/279,617, filed Nov. 15, 2021, entitled “Constructs Comprising Fibrin or Other Blood Products for Meat Cultivation and Other Applications,” by Hosseini, et al., incorporated herein by reference in its entirety. Fibrin is an edible fibrous protein involved in the clotting of blood. It can be formed, for example, by the action of the protease inhibitor thrombin on fibrinogen, which causes it to polymerize and form a clot. Fibrin can be used as a passive scaffolding material in some embodiments. However, in some embodiments, fibrin can specifically bind certain growth factors in the cell culture media that promote cell adhesion, proliferation, and migration. Non-limiting examples include fibronectin, hyaluronic acid, von Willebrand factor, or the like.

In certain embodiments, microcarriers or scaffolds such as those discussed herein may be treated to facilitate binding of cells, such as myoblasts. For example, the microcarriers or scaffolds may be exposed to non-human serum, which may include growth factors that bind to the microcarriers or scaffolds. The growth factors may, for example, promote cell adhesion, proliferation, and/or migration of cells into the microcarriers or scaffolds.

In some embodiments, the microcarriers or scaffolds may comprise any material that forms an edible hydrogel, such as fibrin. For example, in one embodiment, a microcarrier may be formed from a non-human blood plasma, or platelet rich plasma (PRP), both of which contain plasma-rich fibrinogen that can be crosslinked or otherwise processed to form a fibrin hydrogel. Such crosslinking can be achieved by exposure to thrombin, calcium, or other conditions such as those described herein. In some embodiments, fibrin hydrogels are formed using non-human blood plasma, and/or PRP, containing fibrinogen, e.g., at least 10 wt %, or more in some cases.

In certain embodiments, non-human cells such as myoblasts may be seeded on the microcarriers or other scaffolds, and grown in a bioreactor such as described herein. For instance, myoblasts may be grown on microcarriers and, in some embodiments, allowed to differentiate or fuse to form aligned myotubes, e.g., within a bioreactor.

As mentioned, in some cases, the bioreactor may comprise fat or adipose cells. In some cases, the fat may be removed from the bioreactor, and used to form a fat replica, e.g., within an emulsion. An emulsion of fat may be prepared, for example, by emulsifying fat with non-human blood plasma. In some cases, the fat may be caused to form a fat emulsion by mixing the fat with non-human blood plasma. Without wishing to be bound by any theory, it is believed that the plasma has components that can emulsify fat to form fat particles such as chylomicrons. For instance, the plasma may include proteins or surfactants that can from such fat particles. The non-human blood plasma may be treated in some embodiments to form a fat replica. For example, fibrin within the plasma may be caused to clot and/or by causing the fibrin to crosslink, e.g., by exposing it to thrombin, calcium, or other clotting agents such as those described herein. In addition, in some embodiments, a fat replica may comprise a fat emulsion contained within a hydrogel. The hydrogel may be formed from non-human blood plasma, e.g., as discussed, and/or another component. Non-limiting examples of such hydrogels include alginate, gelatin, or others such as those described herein. See, for example, U.S. Ser. No. 63/279,642, filed Nov. 15, 2021, entitled “Systems and Methods of Producing Fat Tissue for Cell-Based Meat Products,” by Hosseini, et al., incorporated herein by reference in its entirety.

In some embodiments, the bioreactor may comprise blood cells. These may be used to provide heme or heme-containing proteins. For instance, in one embodiment, the heme may be obtained from non-human red blood cells, e.g., cultivated within the bioreactor. For example, red blood cells may be lysed, e.g., by exposing the cells to hypoosmotic or distilled water to form a lysate of non-human red blood cells. Red blood cells contain hemoglobin, a structurally similar protein to myoglobin. Hemoglobin also contains a heme moiety. Lysing the red blood cells may release hemoglobin, e.g., into solution. Furthermore, in addition to these, other methods of lysing red blood cells can be used. See, for example, U.S. Ser. No. 63/279,644, filed Nov. 15, 2021, entitled “Production of Heme for Cell-Based Meat Products,” by Hosseini, et al., incorporated herein by reference in its entirety.

As mentioned, some aspects of the present disclosure are generally directed to obtaining cells from an animal, and cultivating the cells within a bioreactor. Thus, some embodiments such as described herein are directed toward repeated blood collection from non-human animals, for example, to obtain blood, blood serum, blood plasma, red blood cells, or other blood products, etc. Non-limiting examples of animals include chicken, cow, pig, sheep, goat, deer, fish, duck, turkey, shrimp, or any other suitable animals. For instance, in some embodiments, blood may be withdrawn from the animal at spaced intervals, so as to allow the animal time to recover and produce new blood. For instance, blood may be withdrawn from the animal every 2 weeks, every 4 weeks, every 6 weeks, every 2 months, or the like. The blood draws may each be processed, for example, as discussed herein. For example, blood cells from the blood may be grown within a bioreactor, e.g., to form a cultivated meat product. In this way, blood can be obtained in certain embodiments in a sustainable and cost-effective manner, and/or without killing the animal. This usage may result, in certain embodiments, in the reduction in carbon emissions, water use, land use, etc.

Thus, certain systems and methods described herein can be useful, for example, by providing meat and other animal derived products for human consumption. Certain embodiments described herein may offer certain advantages as compared to existing agriculture-based methods of meat production, for example, by significantly reducing the number of animals bred for slaughter, thus decreasing the number of foodborne illnesses, diet related diseases, and the incidence of antibiotic resistance and infectious disease (e.g., zoonotic diseases such as Nipah virus and influenza A). In some cases, reducing the number of livestock worldwide may also have an effect on the environmental risks associated with agricultural farming due to, for example, ammonia emissions which contribute significantly to acid rain and acidification of ecosystems. In addition, in some instances, livestock, such as pigs and cows are a major agricultural source of greenhouse gases worldwide. In some embodiments, the structures and methods described herein may allow meat and other animal-derived products to be produced or cultivated in vitro, e.g., using blood and tissue donations obtained from living livestock donors (e.g., not intended for slaughter for human consumption). As a non-limiting example, certain embodiments as described herein are generally directed to a product comprising a muscle replica, a fat replica, and a lysate of red blood cell. Sec, for example, Int. Pat. Apl. Ser. No. PCT/US22/19631, entitled “Methods and Systems of Producing Products such as Animal Derived Products,” filed on Mar. 9, 2022, incorporated herein by reference in its entirety.

The following are each incorporated herein by reference in their entireties: U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; US Provisional Patent Application Ser. No. U.S. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived Products”; U.S. Provisional Patent Application Ser. No. 63/314,171, filed Feb. 25, 2022, entitled “Growth Factors for Laboratory Grown Meat and Other Applications”; and U.S. Provisional Patent Application Ser. No. 63/314,191, filed Feb. 25, 2022, entitled “Methods and Systems of Producing Products Such as Animal Derived Products.”

In addition, U.S. Provisional Patent Application Ser. No. 63/330,824, filed Apr. 14, 2022, entitled “Systems and Methods for Recycling Growth Factors and Other Components,” is incorporated herein by reference in its entirety.

The following examples are intended to illustrate certain embodiments of the present disclosure, but do not exemplify the full scope of the disclosure.

### Example 1

In this example, a bioreactor used to produce a cultivated meat product is demonstrated. After incubation, proliferation, and refilling the bioreactor with cells to be grown to form the cultivated meat product, a first batch analysis of the bioreactor was performed. When the cells within the bioreactor reached 80% confluency, the mixture of media and cells was purified by pumping the reaction mixture out of the bioreactor. Around 50% of the cells were left within the bioreactor for the bioreactor to meet continuous production.

The cell and media mixture medium was then passed through a filter (0.2 micrometer pore size) for cell separation, and the supernatant was collected in a media reservoir (see FIG. 1) and analyzed for growth factors such IGF and FGF, lactic acid, and glucose contents using high-performance liquid chromatography (HPLC) (FIG. 2) using reverse-phase HPLC.

Afterwards, media recovery was performed by ultrafiltration of the media. The used media was pumped through an ultrafilter (DiaCap, Pro, 16H, filter material: 5 kDa, polysulfone, KoA urea (QB=300 ml/min)) at a rate of 60 ml per minute. The membrane was sterile and stable over a range of pH 2-13. The purified media was stored in a stirrer-equipped stainless-steel tank (FIG. 1) and analyzed using HPLC (FIGS. 2 and 3). FIG. 2 illustrates efficacy of media ultrafiltration to maintain the growth factors/proteins concentration, while FIG. 3 illustrates the efficacy of media ultrafiltration to remove the lactic acid. Results from these experiments are shown in Table 1, illustrating the efficacy of media ultrafiltration to remove the lactic acid and maintain the growth factors/proteins concentration.

TABLE 1

| steps | sample | IGF-1 (%) | FGF (%) | Lactic acid (%) |
|---|---|---|---|---|
| 1 | Media before cell culture | 25.1775 | 10.4809 | 1.7413 |
| | (10% FBS) | | | |
| 2 | Media after cell culture | 8.6410 | 9.5344 | 2.3596 |
| 3 | Media after cell culture/ | 15.3981 | 17.2662 | 1.9392 |
| | after microfiltration | | | |
| 4 | Media after | 10.12 | 11.53 | 1.286 |
| | resubstitution of filter | | | |
| | media with basal media | | | |
| | (33%) | | | |

These data show that ultrafiltration was capable of lowering the concentration of waste products such as lactic acid, and substantially preserved the concentration of growth factors.

### Example 2

In this experiment, bovine myoblasts were incubated (100% confluency at the beginning of incubation) in 10% bovine platelet rich plasma (PRP) in DMEM. After 7 days of incubation, the used media was collected. Immediately after collection, the used media was centrifuged at 15,000× for 30 min to remove the particles and avoid filter blockage. The used media was then ultrafiltered using 1 kDa filters to remove various volume used media and topped off with the fresh 10% PRP DMEM to compensate for the lost volume.

The ability of the used media to grow cells was then assessed using bovine myobalsts to determine cell proliferation. The cells were seeded on 24-well plates (15k cells/well), and incubate with 10% PRP-DMEM for 16 hours. Afterwards, the cells were incubated with ultrafiltered media as prepared above.

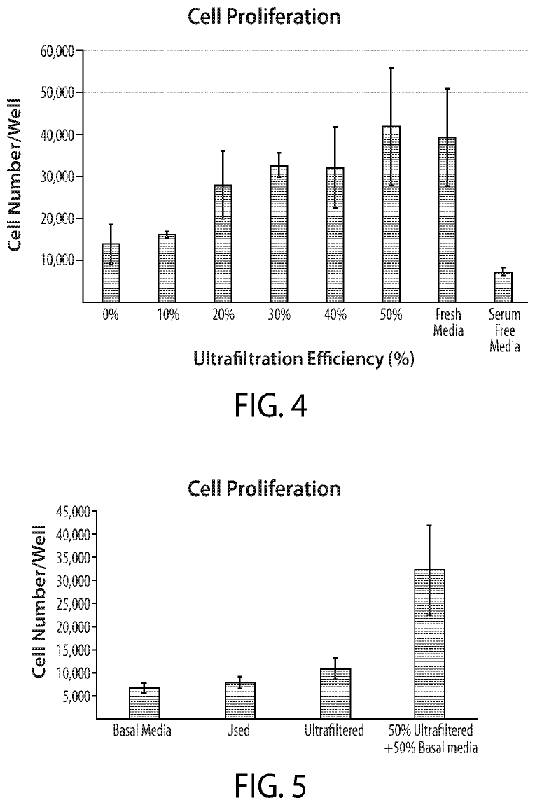

After 48 hours, cell numbers for each well were counted using a cell counter. The results are shown in FIG. 4. It can be seen that the ultrafiltered media was comparable in cell proliferation as fresh media.

In another experiment, the replacement of ultrafiltered media with basal media (without supplementation of 10% PRP) was studied under similar conditions. These results are shown in FIG. 5, showing that supplementation of 50% basal media enhanced cellular proliferation.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either.” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having.” “containing,” “involving.” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method comprising:
separating a fluid from a bioreactor containing a cell-based meat culture, using a first filtration device, into a first retentate and a first filtrate, wherein the first filtrate is substantially free of cells;
separating the first filtrate, using a second filtration device having an average molecular weight cut-off of at least 1 kDa, into a second retentate and a second filtrate;
mixing the second retentate with fresh cell culture media to form a mixture; and
returning the mixture to the bioreactor.

2. The method of claim 1, wherein the fluid within the bioreactor comprises non-human cells.

3. The method of any one of claim 1 or 2, further comprising returning the first retentate to the bioreactor.

4. The method of any one of claims 1-3, wherein the fluid contains a growth factor.

5. The method of any one of claims 1-4, wherein at least 90% of cells entering the first filtration device are retained in the first retentate.

6. The method of any one of claims 1-5, wherein at least 95% of cells entering the first filtration device are retained in the first retentate.

7. The method of any one of claims 1-6, wherein at least 99% of cells entering the first filtration device are retained in the first retentate.

8. The method of any one of claims 1-7, wherein at least 50 mol % of the growth factor in the second retentate is returned to the bioreactor.

9. The method of any one of claims 1-8, wherein at least 80 mol % of the growth factor in the second retentate is returned to the bioreactor.

10. The method of any one of claims 1-9, wherein the first filtration device comprises a tangential flow filter.

11. The method of any one of claims 1-10, wherein the first filtration device has an average pore size of between 0.1 micrometers and 0.3 micrometers.

12. The method of any one of claims 1-11, wherein the second filtration device comprises a tangential flow filter.

13. The method of any one of claims 1-12, wherein the second filtration device has a molecular weight cut-off of at least 1 kDa.

14. The method of any one of claims 1-13, wherein the second filtration device has a molecular weight cut-off between 1 kDa and 5 kDa.

15. The device of any one of claims 1-14, wherein the separator has an average molecular weight cutoff of between 1 kDa and 10 kDa.

16. The method of any one of claims 1-15, wherein the fluid in the bioreactor comprises microcarriers.

17. The method of any one of claims 1-16, wherein the fluid in the bioreactor comprises fat cells.

18. The method of any one of claims 1-17, wherein the fluid in the bioreactor comprises muscle cells.

19. The method of any one of claims 1-18, wherein the fluid in the bioreactor comprises blood cells.

20. The method of any one of claims 1-19, further comprising removing cells from a non-human animal without killing the animal; and adding the cells to the bioreactor to produce the cell-based meat culture.

21. A method comprising:
separating a fluid from in a bioreactor, using a first filtration device, into a first retentate and a first filtrate, wherein the first filtrate is substantially free of cells;
separating the first filtrate, using a second filtration device having an average molecular weight cut-off of at least 1 kDa, into a second retentate and a second filtrate; and
mixing the second retentate with fresh cell culture media to form a mixture.

22. The method of claim 21, further comprising returning the mixture to the bioreactor.

23. The device of any one of claim 21 or 22, wherein the separator has an average molecular weight cutoff of between 1 kDa and 10 kDa.

24. A device, comprising:
a bioreactor;
a first fluidic connection connecting the bioreactor to an inlet of a first filtration device, the first filtration device further comprising a first retentate outlet and a first filtrate outlet;
a second fluidic connection connecting the first filtrate outlet to the inlet of a second filtration device comprising a separator having an average molecular weight cutoff of at least 1 kDa, the second filtration device further comprising a second retentate outlet and a second filtrate outlet;
a third fluidic connection connecting the second retentate outlet with a first inlet of a mixing device, the mixing device further comprising a second inlet and an outlet; and
a fourth fluidic connection connecting the outlet of the mixing device with the bioreactor.

25. The device of claim 24, further comprising a fifth fluidic connection connecting a source of cell culture media to the second inlet of the mixing device.

26. The device of any one of claim 24 or 25, wherein the first filtration device comprises a tangential flow filtration device.

27. The device of any one of claims 24-26, wherein the second filtration device comprises a tangential flow filtration device.

28. The device of any one of claims 24-27, wherein the separator has an average molecular weight cutoff of between 1 kDa and 10 kDa.

29. A method comprising:
removing fluid comprising non-human cells and growth factors from a bioreactor containing a cell-based meat culture;
separating the non-human cells from the fluid;
returning the non-human cells to the bioreactor;
separating the growth factors from the fluid; and
returning the growth factors to the bioreactor.
